(19) United States
(12) Patent Application Publication
(10) Pub. No.: US 2012/0052035 A1
(43) Pub. Date: Mar. 1, 2012

(54) Two Phase Depilatory Composition

(76) Inventors: Lauren Ciemnolonski, Princeton, NJ (US); Steven T. Adamy, Lawrenceville, NJ (US)

(21) Appl. No.: 13/221,220

(22) Filed: Aug. 30, 2011

Related U.S. Application Data
(60) Provisional application No. 61/378,309, filed on Aug. 30, 2010.

(51) Int. Cl. A61K 8/72; A61Q 9/04

(52) U.S. Cl. 424/70.11

(57) ABSTRACT

An improvement on the prior art dual phase chemical depilatory composition. The present invention is a dual phase present composition comprising: (1) a polymer; (2) depilatory active ingredients; (3) complexing agent; and (4) polyol.

### CROSS-REFERENCE TO RELATED APPLICATION

This application is related to U.S. Provisional Patent Application Ser. No. 61/378,309 filed Aug. 30, 2010 and takes priority therefrom.

### FIELD OF THE INVENTION

The invention relates to depilatory compositions which can be peeled away from the skin.

### BACKGROUND OF THE INVENTION

As an alternative to shaving, many hair removal products available today perform through either chemical degradation (depilation) or mechanical removal (epilation) of the hair. Compositions for removing superfluous body hair are well known and are of various types. Currently on the market there are many different types of chemical depilatories and epilation products. Epilatory products, such as waxing, require initial heating before being applied to the skin in a generally molten state. It is then allowed to solidify before being removed from the skin together with unwanted hair.

One major drawback with waxing is the pain associated with hair removal. Waxing works by pulling the hair out from the roots. The hair follicle is surrounded by a small muscle that allows the hair to be erected in response to cold or excitement. Also attached with this muscle are nerve endings.

These structures make pulling the hair out fairly hard and painful. The pain creates a fear associated with the waxing procedure which will cause many people not to repeat the procedure. Even if the person does repeat the waxing, the fear can cause the muscles to tense up, which in turn creates even more pain in subsequent procedures.

Chemical-based depilation offers an attractive alternative to waxing or shaving with the removal of hair being accomplished through the cleavage of disulfide bonds in the hair fiber as well as denaturation of the associated protein matrix. Unlike waxing, chemical-based depilation digests the hair and the hair is not pulled out, thus avoiding the pain associated with waxing. However, a relatively small proportion of individuals, compared with shaving, employ depilatories. One reason for the limited use may be that the hair cleavage reaction must be run at a very high pH. A typical example is the Nair® lotion product, having a pH of about 12.5 to 13.

Chemical-based depilatory compositions typically use a thiol-based depilatory agent, such as thioglycolic acid, for removal of unwanted body and facial hair and its use is well established in the art. These agents react by reducing hair's protein disulfide bonds to sulfhydryl anions, thereby allowing easy removal of the weakened hairs when washed or wiped away. However, in using thiols, it was discovered that certain conditions facilitated the effectiveness of this reaction. One such condition is high alkalinity to provide ionized reactants. Not only does the high pH (approximately 12.0-13) result in ionized thiols, but they also result in increased penetration of a reactant. Substances to provide further enhancement of penetration by active thiols were developed.

Currently on the market there are many different types of chemical depilatories. They range in form from creams to gel to aerosol mousses and spray products. Depilatory composition in the form of a cream is applied to the skin at room temperature. One drawback to many of these depilatory products is the required clean up. As discussed earlier, chemical depilatories contain a strong alkali, usually a metal hydroxide. In addition, there is usually a reducing agent used. These chemicals can cause considerable damage to the skin if not properly removed. Likewise, they will quickly cause blindness if they get into the eyes. Product wiped off the skin is still active and will corrode aluminum and many organic materials, creating the possibility of property damage or injury to the user, children or pets through inadvertent contact.

Products have been made to help reduce the risks associated with using these high pH depilatory products by trapping the dangerous chemicals in a solid matrix, i.e., a semi-solid hydrogel, after use, thereby simplifying disposal because there will not be liquid which can stick to surfaces. These depilatory products form a dry rubbery film on the skin which after a period of a few minutes can be peeled in one piece from the skin. In this manner, the product is similar to a hot wax depilation. After removal of the product, nothing save a very slight residue is left behind, which could be removed with a damp towel. However, unlike wax, the hair will have been digested by chemicals and not pulled out, thereby avoiding the pain associated with pulling out hair as in waxing. Further, there is the elimination of a great deal of mess. U.S. Patent Application Publication No. 2009/0087499 describes such a product.

U.S. Patent Application Number 2009/0087499 describes a dual phase depilatory composition that forms a dry rubbery film on the skin which after a period of a few minutes can be peeled in one piece from the skin. In particular, the '499 invention comprises a solvent phase (water based), a polymer/s capable of creating a hydrosol and complexing to form a hydrogel (polyvinyl alcohol being the most preferred polymer), depilatory active ingredients, complexing agent/active ingredient delivery system (borax being the most preferred complexing agent), a filler and/or gel strengthening agents, a skin protecting agent, odor masking agents and fragrance, and optionally chelating and colorant materials.

To form a dry rubbery film on the skin, it is widely known that polyvinyl alcohol (PVOH) will crosslink in the presence of borate ions to form a semi-solid hydrogel. The mechanism as described by R. G. Loughlin et al., European Journal of Pharmaceutics and Biopharmaceutics, 69 (2008) 1135-46 is shown below:

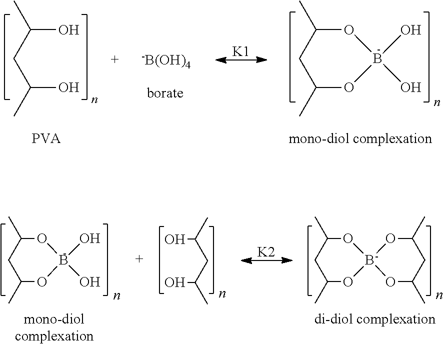

The polymer and crosslinking agent are kept in separate phases to prevent the crosslinking reaction from starting until the two phases are mixed together on the desired area of skin and the hydrogel can form. However, once the two phases are mixed, this cross-linking reaction occurs rapidly when sufficient quantities of the borate ion are present and the reaction needs to be delayed to give the consumer adequate time to blend the two phases, apply to the skin, and allow depilation to occur.

To overcome the issue of the rapid cross-linking reaction once the two phases are mixed, the prior art '499 patent application encapsulates the cross-linking/complexing agent. If the complexing agent is not encapsulated, then the reaction will occur too quickly and not allow proper mixing and dispersion before gelling. Several different encapsulates including cellulose, enteric coating and combinations may be used to encapsulate the complexing agent, and the most preferred coat is stearic acid.

However, there are problems associated with encapsulating the complexing agent. Encapsulation requires another processing step which increases the costs associated with preparing the depilatory composition and increases the opportunities for process failures. For example, in the '499 application, borax (the complexing agent) is encapsulated with stearic acid by melting stearic acid and rapidly stirring the finely crushed complexing agent into it. While borax was suspended in the melted stearic acid, the mixture was sprayed at high pressure in the same manner as spray drying. Both melting the stearic acid and spray drying the borax/stearic acid particles can significantly increase the cost of production of the depilatory composition.

It would be most useful to formulate a depilatory composition as a dual phase depilatory composition that forms a semi-solid hydrogel, but in such a way that the cross-linking agent does not have to be encapsulated in order to delay the cross-linking reaction.

### SUMMARY OF THE INVENTION

The present invention is an improvement on the prior art dual phase chemical depilatory composition because the present invention avoids the need to encapsulate the cross-linking agent. It has been found that polyols could be used in a dual phase chemical depilatory composition to help mediate the cross-linking reaction and give a consumer adequate time to blend the two phases, apply the composition to the skin, and allow depilation to occur prior to the solidification of the polymer.

### DETAILED DESCRIPTION OF THE INVENTION

The present invention is an improvement on the prior art dual phase chemical depilatory composition. The prior art dual phase chemical depilatory composition described in U.S. Patent Application Publication No. 2009/0087499 has a solvent phase (water based), a polymer/s capable of creating a hydrosol and complexing to form a hydrogel (polyvinyl alcohol being the most preferred polymer), depilatory active ingredients, complexing agent/active ingredient delivery system (borax being the most preferred complexing agent), a filler and/or gel strengthening agents, a skin protecting agent, odor masking agents and fragrance, and optionally chelating and colorant materials. A dual phase system is used in order to keep the two phases separate and until they are mixed immediately prior to use in order to prevent the crosslinking reaction from starting until needed because over time because the complexing agent will cause the polymer to crosslink and turn to rubber. Because of these reasons, the product must be split into two separate phases that are kept separated until used. Even though the two phases are kept separate until use, one major issue in formulating a polymer and a complexing agent is that cross-linking reaction occurs very rapidly when sufficient quantities of a complexing agent are used and the polymer forms a hydrogel before the user is able to adequately apply the depilatory composition to the affected area. To overcome the issue of the quick reaction time between the complexing agent and the polymer, the complexing agent in the '499 patent application is further encapsulated to allow proper mixing and dispersion before gelling. However, due to the issues that arise from encapsulation, such as the increased costs associated with encapsulation, a better formulation is needed.

The present invention is an improvement on the prior art dual phase chemical depilatory composition because the present invention avoids the need to encapsulate the cross-linking agent. It has been found that polyols could be used in a dual phase chemical depilatory composition to help mediate the cross-linking reaction and give a consumer adequate time to blend the two phases, apply the composition to the skin, and allow depilation to occur. According to R. G. Loughlin et al., European Journal of Pharmaceutics and Biopharmaceutics, 69 (2008) 1135-46, polyols help mediate the cross-linking reaction by competing with the PVOH to complex with the borate ions. Use of polyols in the present invention helps avoid the need to encapsulate the cross-linking agent and thus avoids the increased costs associated with encapsulation.

In addition to avoiding the costs associated with encapsulation, it has been found that using polyols in a two-phase depilatory composition is a much more controllable method to mediate the crosslinking reaction compared to encapsulation. The encapsulation method is very dependent on the encapsulation process, i.e., encapsulation material, coating thickness, etc. The use of polyol rather than encapsulation also avoids the issue of having to ensure the encapsulated material is evenly dispersed throughout the formulation. Prior art depilatory compositions that employing encapsulation uses structuring agents and surfactants to ensure even distribution throughout the formulation.

Polyol can also act as a humectant in the formulation and can slightly adjust the rheology of the system in a beneficial way.

The present invention is based on the use of thiol-based depilatory agents for hair removal and is maintained in a dual phase system. The two phases are kept separate and mixed immediately prior to use in order to prevent the crosslinking reaction from starting until needed because over time, the complexing agent will cause the polymer to complex and turn to rubber. Because of these reasons, the product must be split into two separate phases that are kept separated until used. There are several key components to the dual phase present composition: (1) a polymer phase containing a polymer capable of creating a hydrosol and complexing to form a hydrogel; (2) a complexing agent phase containing a complexing agent capable of complexing the polymer into a hydrogel; (3) depilatory active ingredients; and (4) polyol. Other ingredients that can be used in the present invention include the following: pH buffers; emulsifiers; thickeners and/or gel strengthening agents; a skin protecting agent; humectant and/or moisturizer; surfactants; odor masking agents and fragrance; chelating materials; and colorant materials.

### Polymer/s

The composition of the present invention includes one or a combination of monomers, macromers, and/or polymers that polymerize or otherwise thicken upon or shortly after contact with a complexing agent to form a hydrogel. The hydrogel further desirably includes a solvent, desirably aqueous. All of the composition ingredients should be biocompatible or non-irritant in the amounts present in the final hydrogel composition.

The invention contains a polymer which is able to complex or crosslink upon treatment with a suitable compound. The effect of this complexation or crosslinking creates a hydrogel from the hydrosol of the polymer. The hydrogel is tough and resistant to breakage, allowing the gel to be removed in one piece from the skin.

The hydrogel can be made from one or more polymers, also referred to as macromers. Macromers include a hydrophilic or water soluble region and one or more crosslinkable regions. The macromers may also include other elements such as one or more degradable or biodegradable regions. A variety of factors—primarily the desired characteristics of the formed hydrogel determines the most appropriate macromers to use. The basic requirements for the macromers are biocompatibility and the capacity to be applied to the desired area whereupon it forms a gel. Many macromer systems that form biocompatible hydrogels can be used.

Macromers can be constructed from a number of hydrophilic polymers, such as, but not limited to, polyvinyl alcohols (PVA), polyethylene glycols (PEG), polyvinyl pyrrolidone (PVP), polyalkyl hydroxy acrylates and methacrylates (e.g. hydroxyethyl methacrylate (HEMA), hydroxybutyl methacrylate (HBMA), and dimethylaminoethyl methacrylate (DMEMA)), polysaccharides (e.g. cellulose, dextran), polyacrylic acid, polyamino acids (e.g. polylysine, polyethylimine, PAMAM dendrimers), polyacrylamides (e.g. polydimethylacrylamid-co-HEMA, polydimethylacrylamid-co-HBMA, polydimethylacrylamid-co-DMEMA). The macromers can be linear or can have a branched, hyperbranched, or dendritic structure.

The macromers include two or more crosslinkable groups. Crosslinking of macromers may be via any of a number of means, such as physical crosslinking or chemical crosslinking. Physical crosslinking includes, but is not limited to, complexation, hydrogen bonding, desolvation, Van der waals interactions, and ionic bonding. Chemical crosslinking can be accomplished by a number of means including, but not limited to, chain reaction (addition) polymerization, step reaction (condensation) polymerization and other methods of increasing the molecular weight of polymers/oligomers to very high molecular weights. Chain reaction polymerization includes but is not exclusive to free radical polymerization (thermal, photo, redox, atom transfer polymerization, etc.), cationic polymerization (including onium), anionic polymerization (including group transfer polymerization), certain types of coordination polymerization, certain types of ring opening and metal metathesis polymerizations, etc. Step reaction polymerizations include all polymerizations which follow step growth kinetics including but not limited to reactions of nucleophiles with electrophiles, certain types of coordination polymerization, certain types of ring opening and metal metathesis polymerizations, etc. Other methods of increasing molecular weight of polymers/oligomers include but are not limited to polyelectrolyte, formation, grafting, ionic crosslinking, etc.

In one embodiment, the hydrogel is formed from macromers having a backbone of a polymer comprising units having a 1,2-diol or 1,3-diol structure, such as polyhydroxy polymers. For example, polyvinyl alcohol (PVA) or copolymers of vinyl alcohol contain a 1,3-diol skeleton. The backbone can also contain hydroxyl groups in the form of 1,2-glycols, such as copolymer units of 1,2-dihydroxyethylene. These can be obtained, for example, by alkaline hydrolysis of vinyl acetate-vinylene carbonate copolymers. Other polymeric diols can be used, such as saccharides.

The macromers have at least two pendant chains containing groups that can be crosslinked. The term group includes single polymerizable moieties, such as an acrylate, as well as larger crosslinkable regions, such as oligomeric or polymeric regions. The crosslinkers are desirably present in an amount of from approximately 0.01 to 10 milliequivalents of crosslinker per gram of backbone (meq/g), more desirably about 0.05 to 1.5 meq/g. The macromers can contain more than one type of crosslinkable group.

The pendant chains are attached via the hydroxyl groups of the polymer backbone. Desirably, the pendant chains having crosslinkable groups are attached via cyclic acetal linkages to the 1,2-diol or 1,3-diol hydroxyl groups.

In one embodiment, the compositions include modified polyvinyl alcohol (PVA) macromers, such as those described in U.S. Pat. Nos. 5,508,317, 5,665,840, 5,849,841, 5,932,674, 6,011,077, 5,939,489, or 5,807,927. The macromers disclosed in U.S. Pat. No. 5,508,317, for example, are PVA prepolymers modified with pendant crosslinkable groups, such as acrylamide groups containing crosslinkable olefinically unsaturated groups. These macromers can be polymerized by photopolymerization or redox free radical polymerization, for example.

The hydrophobicity of these macromers can be increased by substituting some of the pendant hydroxyl groups with more hydrophobic substituents. The properties of the macromers, such as hydrophobicity, can also be modified by incorporating a comonomer in the macromer backbone. The macromers can also be formed having pendant groups crosslinkable by other means.

The preferred polymer for use has been found to be polyvinyl alcohol. There are several grades of polyvinyl alcohol available. They differ in the degree of hydrolysis performed on the polyvinyl acetate starting stock. Most commercial grades vary between 85-99%. The level of PVOH in the final formulation may be in the range of 1 to 50 wt. % with the preferred range of 5 to 20 wt. %.

### Depilatory Active Ingredients

The depilatory agent is a substance capable of degrading keratin. The active ingredients are used in order to denature the proteins in the hair causing it to swell by disrupting the hydrogen bonding in the protein chain. This disruption of the hydrogen bonding will open the protein up for easier attachment of the peptide bonds by hydroxide ions. The depilatory agent may be, for example, a sulfur compound such as potassium thioglycolate, dithioerythritol, thioglycerol, thioglycol, thioxanthine, thiosalicylcic acid, N-acetyl-L-cysteine, lipoic acid, NaHSO₃, Li₂S, Na₂S, K₂S, MgS, CaS, SrS, BaS, (NH₄)₂S, sodium dihydrolipoate 6,8-dithiooctanoate, sodium 6,8-dithiooctanoate, salts of hydrogen sulfide for example NaSH or KSH, thioglycolic acid, thioglycerol, 2-mercaptopropionic acid, 3-mercaptopropionic acid, thiomailic acid, ammonium thioglycolate, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazide, keratinase, hydrazine sulphate, hydrazine disulphate, triisocyanate, guanidine thioglycolate, hair reducing pack, calcium thioglycolate and/or cysteamine. Preferably, a depilatory composition comprises cysteamine.

Suitably, a depilatory agent is provided in the depilatory composition in an amount of between 1 wt % and 10 wt %, preferably between 1.5 wt % and 8 wt % and more preferably between 2 wt % and 6 wt % of the total weight of the final mixed composition.

### Complexing Agent

A complexing agent is used in the present invention to complex or crosslink the polymer. The effect of this complexation or crosslinking creates a hydrogel from the hydrosol of the polymer.

If PVOH is used as the polymer in the present invention, the complexing agent is preferably a boron containing compound having free hydroxyl groups attached to the boron atoms, such as commercial boric acid (ortho boric acid, H₃BO₃ and its hydrated forms H₃BO₃.H₂O) and borax (sodium tetraborate decahydrate, Na₂B₄O₇.10H₂O and other hydrate and anhydrous forms). Suitable borates for crosslinking include the alkali metal and alkaline earth metal borates, ammonium borates and amine borates. These include, without limitation, ammonium borate, NH₄HB₄O₇ 3H₂O; calcium metaborate, Ca(BO₂)₂; calcium metaborate hexahydrate, Ca(BO₂)₂ 6H₂O; calcium tetraborate, CaB₄O₇; lithium metaborate, LiBO₂; lithium tetraborate, Li₂B₄O₇ 5H₂O; potassium metaborate, K₂B₄O₇ 5H₂O; sodium metaborate, Na₂B₂O₄ and the tetrahydrate thereof, Na₂B₂O₄ 4H₂O; sodium tetraborate, Na₂B₄O₇, the penta hydrate thereof, Na₂B₄O₇ 5H₂O and the decahydrate thereof, Na₂B₄O₇ 10H₂O; methylammonium hydrogen tetraborate, NH₃CH₃HB₄O₇; dimethylammonium hydrogen tetraborate, NH₂(CH₃)₂HB₄O₇; and the like. Borax is an effective gelling agent for PVOH and is the most preferred complexing agent when PVOH is used as the polymer. The level of the borate ion salt in the final mixed formulation may be in the range of 0.0001 to 10 wt. % with preferred levels in the range of 0.01 to 2%.

### Polyol

The crosslinking reaction occurs rapidly in the polymer when sufficient quantities of the complexing agent are present. While it was found that very low concentrations of the complexing agent will delay the reaction, this however also weakens the hydrogel that is eventually formed. For purposes of the depilatory product, a robust hydrogel is required to help with the removal of depilated hair. Thus, the borate ion concentration needs to be sufficient yet the reaction needs to be delayed to give the consumer adequate time to blend the two phases, apply to the skin, and allow depilation to occur.

The polyols of this invention are defined in one non-limiting embodiment as polyols having at least one hydroxyl group on two adjacent carbon atoms. The adjacent carbon atoms may have more than one hydroxyl group, and the polyol may have more that two adjacent carbon atoms, each having at least one hydroxyl group. In another embodiment of the invention, the polyols are monosaccharides, which are glycerols (trihydric monosaccharides having three hydroxyl groups) and sugar alcohols (having more than three hydroxyl groups) and oligosaccharides. In another embodiment of the invention, the polyols may have one of the following formulae:

CH2OH—(CHOH)n—CH2OH (I)

HC═O—(CHOH)n—CH2OH (II)

CH2OH—C═O—(CHOH)n—CH2OH (III)

where n is from 2 to 5, and the hydroxyls may be in the cis or trans orientation. In another embodiment of the invention, the polyols are acids, acid salts, fatty acids (alkyl glycosides), and alcohol, alkyl and amine derivatives (glycosylamines) of monosaccharides and oligosaccharides. Specific examples of polyols falling within these definitions include, but are not necessarily limited to, mannitol (manna sugar, mannite), sorbitol (D-sorbite, hexahydric alcohol), xylitol, glycerol, glucose, (dextrose, grape sugar, corn sugar), fructose (fruit sugar, levulose), maltose, lactose, tagatose, psicose, galactose, xylose (wood sugar), allose (.beta.-D-allopyranose), ribose, arabinose, rhamnose, mannose, altrose, ribopyranose, arabinopyranose, glucopyranose, gulopyranose, galatopyranose, psicopyranose, allofuranose, gulofuranose, galatofuranose, glucosamine, chondrosamine, galactosamine, ethyl-hexo glucoside, methyl-hexo glucoside, aldaric acid, sodium aldarate, glucaric acid, sodium glucarate, gluconic acid, sodium gluconate, glucoheptonic acid, sodium glucoheptonate, and mixtures thereof. In one non-limiting embodiment of the invention, the molecular weight of the simple polyols may range from about 65 to about 500, where an alternate embodiment for the molecular weight ranges from about 90 to about 350. Useful oligosaccharides may have molecular weights ranging from about 450 to about 5000 in one non-limiting embodiment, with most ranging from about 480 to about 1000 in another non-limiting embodiment.

In general, the polyol used in the present invention depends on the polymer and/or complexing agent used. The polyol may be but is not limited to, for example, malititol (C12H24O11), dulitol (C6H14O6), D-Mannitol (C6H14O6), D-Sorbitol (C6H14O6), xylitol (C5H12O5), meso-erythritol (C4H12O4), 1,2-propandiol (C3H8O2), Propan-2-ol (C3H8O), and glycerol (C3H8O3). The preferred polyols are mannitol and sorbitol. The level of polyol in the final mixed formulation may be in the range of 0.1 to 10 wt. % with the preferred range of 0.1 to 1 wt. %.

### Other Ingredients

### pH Buffers

The depilatory composition of the present invention is alkaline, containing a salt to buffer the system at a higher pH. Examples of pH buffers include calcium hydroxide and sodium hydroxide. Due to the characteristics of the pH buffer, the depilatory composition maintains an alkaline pH. An alkaline environment is highly beneficial for the hair removal process. The mixture of the two phases results in a mixed composition with a pH high enough to cause effective depilation, but low enough to minimize irritation. Desirably the pH of the composition of the present invention is at least 12.

### Emulsifiers

Emulsifiers may be used in the present invention. The formula does not need to create a long term stable emulsion, just one stable to high electrolytes for a few hours to days. In theory, one of the phases should carry the active ingredients until mixed with the second phase, upon which it will self emulsify and allow the depilatory agent, polymer, and complexing agent to dissolve and mix. Once dissolved, the actives will begin the process of digesting the hair.

### Thickeners/Gel Strengthening Agents

Among the other ingredients useful in these various embodiments is a gelling agent or thickener, present at levels of from about 0% to about 30%. The thickeners used could include both natural and synthetic ones such as tragacanth, xanthan, karaya, and guar gums, clays, methyl or hydroxyethyl cellulose, hydroxypropyl cellulose, carboxymethyl cellulose, fatty and polyvinyl alcohols, modified starches and sugars, and mixtures thereof. Emollients such as paraffin, petrolatum, mineral oil, fatty alcohols, silicone oils, and mixtures thereof present at levels of from about 0% to about 60%, can also be included.

Materials can also be added to the formula to increase the strength of the set hydrogel. These have been a variety of materials. One of the most common and useful is kaolin clay. Kaolin is low in free cations.

Silica has been used to give the gel strength. Embodiments have included fumed silica and surface treated silicas.

Other agents can be used to control the properties of the hydrosol before gelling or to suspend the powder ingredients. These can be other polymers such as Carbomer or Xanthan gums. Pure viscosity control could be achieved through the use of non-ionic celluloses such as HEC or CMC. These will hydrolyze in the final mixture but may provide temporary needed viscosity control.

Specialty polymers that thicken on increased pH such as Stutture 2001 from national starch may be of benefit.

### Skin Protecting Agent

As with all depilatories a skin protecting agent should be added to the formula. Most preferred is to use dimethicone for this function. It provides and occlusive barrier that can help to protect the skin from the caustic active ingredients. 0.5-2.0% should be sufficient. It is important to not use excessive amounts of a skin protestant like dimethicone or petrolatum because they can coat the hair preventing attach of the protein chain.

### Humectant/Moisturizers

The depilatory composition of the present invention may additionally include one or more oils. The oil may act as a moisturizer and/or humectant. Suitable oils include allantoin, shea butter, cocoa butter, goa butter, kukui nut oil, coconut oil, castor oil, palm oil, olive oil, avocado oil, apricot kernel oil, sweet almond oil and hemp oil. Other oils include mineral oils (eg. paraffin oil), isohexane and sunflower seed oil. Preferably, the composition includes mineral oil.

The oil may be present in the composition in an amount 0.01% to about 1.5% by weight of the composition, preferably in an amount less than about 1.0% by weight, further preferably less than about 0.1% by weight of the composition.

### Surfactants

The depilatory composition of the present invention may contain surfactants. The surfactant may be anionic, cationic or non-ionic, however it is preferred that the surfactant is non-ionic. Examples of suitable surfactants include cetearyl phosphate, cetearyl alcohol, stearyl ether, cetearyl alcohol, cetearyl glycoside, cetostearyl alcohol and/or ceteareth 20.

The one or more surfactant is preferably present in an amount less than 4% by weight of the composition, further preferably less than about 3.8% by weight, such as around 3.5% by weight of the composition.

Preferably, when the composition includes ceteareth 20 it is present in an amount of not more than about 1.0% by weight of the composition, such as less than about 0.9% by weight of the composition.

Preferably, when composition includes stearyl ether it is present in an amount of less than 0.8% by weight of the composition, further preferably less than about 0.6% by weight of the composition.

Preferably, when the composition includes cetearyl alcohol it is present in an amount of less than 2.5% by weight of the composition, further preferably less than about 2.3% by weight of the composition. It is particularly preferred that the cetearyl alcohol is present in an amount less than about 2.2% by weight of the composition.

### Odor Masks and Fragrances

Although not necessary to the formulation special attention must be paid to the fragrances. Most fragrances are not stable to high pH systems. Some fragrances at this pH seem to cause a crosslinking or complexing with the polyvinyl alcohol because on their addition the polymer phase quickly turns to rubber.

### Chelating Material

The addition of ethylenediaminetetraacetic acid (EDTA) is vitally important because thioglycollates will complex with any free iron and turn a purple color. EDTA salts should be added to both phases.

### Colorants

Colorants are not necessary to the formulation but may be used for commercial appeal.

### Dual Phase System

The depilatory composition of the present invention is maintained in a dual phase system. The two phases are kept separate and mixed immediately prior to use. The advantage of keeping the two phases separate stems from preventing the crosslinking reaction from starting until needed. In addition, in order to have a stable product in a container, all of the ingredients cannot be placed together. Over time, cations will cause the polymer, such as PVOH, to complex and turn to rubber. Because of these reasons, the product must be split into two separate phases that are kept separated until used.

### Polymer Phase

The first phase contains the polymer, but can also contain water, depilatory active, thickeners, structuring agents, humectants and polyols. This polymer phase should be mostly water to assure good dissolution of the polymer. Any ingredient sensitive to alkaline hydrolysis must be avoided unless the hydrolysis end products are favorable to the product function and non-irritating.

### Complexing Agent Phase

The second phase contains the complexing agent, but can also contain polyols, water, depilatory active, thickeners, structuring agents and humectant.

### Dispensing Container

The dispensing container must be capable of holding and thereafter dispensing the two phases of the product in the correct proportions. Most preferably the dispenser will have a disposable mixing tip to facilitate the complete mixture of the phases before the application to the skin.

In one embodiment, a double plunger system is used. In another dual product chambers in combination with a pump system is employed.

The following are illustrative examples of a dual phase hair removal composition in accordance with the present invention. These examples are not intended, however, to limit or restrict the scope of the invention in any way and should not be construed as identifying specific materials, parameters or ranges which must be utilized exclusively in order to practice the present invention.

### Example

With the necessary and optional ingredients thus described, an exemplary embodiment of the depilatory composition of the present invention, with each of the components set forth in weight percent, are shown in the tables below. Table 1 shows the components of the polymer phase of the present invention.

TABLE 1
Polymer phase

| Component | Wt. % |
|---|---|
| Deionized Water | 52.683 |
| Cysteamine Hydrochloride | 8.499 |
| Ca(OH)₂ | 23.515 |
| Cetearyl Alcohol & Ceteareth-20 | 4.249 |
| Red Iron Oxide | 0.006 |
| Light Mineral Oil | 6.799 |
| PVOH (87% hydrolyzed, 146- | 4.249 |
| 186K molecular weight) | |
| Sum | 100 |

To make the polymer phase composition described in Table 1, the following procedure was used. The polymer phase in Table 1 was made by first adding water to a beaker and began mixing. Next, the depilatory agent, cysteamine HCl, was added to the beaker. Once the depilatory agent was added, then the Ca(OH)₂ was added to the mixture. The mixture was then heated to 75-80° C.

Once the mixture reached the desired temperature, the mixer was stopped and the cetearyl alcohol and ceteareth-20 was added to the beaker. Dye was added on top of the unmelted wax and mixing was started again until all the wax was dissolved. The mixture was stopped to check to see that all the wax had been dissolved. Once dissolution of all the wax was confirmed, the mixing was again stared and mineral oil added to the mixture. While the mixing continued, the heat was turned off and let cool to room temperature. Once the desired temperature was reached, the polymer PVOH solution was added to the mixture and mixed for five minutes. The pH was then measured to ensure a minimum pH of 12 is reached. If the pH was too low, more Ca(OH)₂ was added and recorded.

Table 2 shows the components of the complexing agent phase of the present invention.

TABLE 2
Complexing agent phase

| Component | Wt. % |
|---|---|
| Deionized Water | 65.893 |
| Cysteamine Hydrochloride | 9.933 |
| Ca(OH)2 | 10.608 |
| Cetearyl Alcohol & Ceteareth-20 | 4.966 |
| Yellow Iron Oxide | 0.008 |
| Light Mineral Oil | 7.946 |
| Borax | 0.149 |
| D-sorbitol | 0.497 |
| Sum | 100 |

To make the complexing agent phase composition described in Table 2, the following procedure was used. The complexing agent phase in Table 2 was made by first adding water to a beaker and began mixing. Next, the depilatory agent, cysteamine HCl, was added to the beaker. Once the depilatory agent was added, then the Ca(OH)₂ was added to the mixture. The mixture was then heated to 75-80° C.

Once the mixture reached the desired temperature, the mixer was stopped and the cetearyl alcohol and ceteareth-20 was added to the beaker. Dye was added on top of the unmelted wax and mixing was started again until all the wax was dissolved. The mixture was stopped to check to see that all the wax had been dissolved. Once dissolution of all the wax was confirmed, the mixing was again stared and mineral oil added to the mixture. While the mixing continued, the heat was turned off and let cool to room temperature. Once the desired temperature was reached, the borate salts and remaining ingredients were added to the mixture and mixed for five minutes. The pH was then measured to ensure a minimum pH of 12 is reached. If the pH was too low, more Ca(OH)₂ was added and recorded.

Table 3 shows the components of the equal combination by weight and/or volume of the polymer phase and complexing agent phase of the present invention.

TABLE 3
Composition of combined phases

| Component | Wt. % |
|---|---|
| Deionized Water | 58.773 |
| Cysteamine Hydrochloride | 9.160 |
| Ca(OH)2 | 17.564 |
| Cetearyl Alcohol & Ceteareth-20 | 4.580 |
| Red Iron Oxide | 0.003 |
| Light Mineral Oil | 7.328 |
| PVOH (87% hydrolyzed, 146- | 2.290 |
| 186K molecular weight) | |
| Yellow Iron Oxide | 0.004 |
| Borax | 0.069 |
| D-sorbitol | 0.229 |

To remove unwanted hair from a particular body area, the two phases of the depilatory composition of the invention are applied to the skin surface of the hairy area to form a coating. After waiting for a sufficient period of time for the active depilatory ingredient to “digest” or break down the hair shafts and for a hydrogel to form, typically 5-15 minutes, the hydrogel, which contains the digested hair, is peeled off the skin and discarded.

Whereas particular embodiments of the present invention have been described for the purposes of illustration, variations may suggest themselves to those skilled in the art without departing from the invention as claimed.

1. A dual phase depilatory composition comprising:
a) a polymer phase comprising a polymer capable of creating a hydrosol and complexing to form a hydrogel;
b) a complexing agent phase comprising a complexing agent that complexes said polymer and creates a hydrogel from the hydrosol of said polymer;
c) a depilatory active ingredient; and
d) polyol.

2. The composition of claim 1 wherein said polymer is polyvinyl alcohol.

3. The composition of claim 2 wherein said polymer is 1 to 50 wt. % of the overall depilatory composition.

4. The composition of claim 2 wherein said polymer is 5 to 20 wt. % of the overall depilatory composition.

5. The composition of claim 1 wherein said polymer phase and said complexing agent phase are kept apart until use.

6. The composition of claim 1 wherein said depilatory active ingredient is cysteamine hydrochloride.

7. The composition of claim 6 wherein said cysteamine hydrochloride is 1 to 10 wt. % of the overall depilatory composition.

8. The composition of claim 1 wherein said complexing agent is a boron containing compound.

9. The composition of claim 8 wherein said complexing agent is borax.

10. The composition of claim 9 wherein said borax is 0.0001 to 10 wt. % of the overall depilatory composition.

11. The composition of claim 9 wherein said borax is 0.01 to 2 wt. % of the overall depilatory composition.

12. The composition of claim 1 wherein said polyol is mannitol or sorbitol.

13. The composition of claim 1 wherein said polyol is sorbitol.

14. The composition of claim 12 wherein said polyol is 0.1 to 10 wt. % of the overall depilatory composition.

15. The composition of claim 12 wherein said polyol is 0.1 to 1 wt. % of the overall depilatory composition.

16. The composition of claim 1 further comprising a pH buffer to provide a pH of at least 12.0.

17. The composition of claim 16 wherein said pH buffer is calcium hydroxide.

18. The composition of claim 1 wherein said depilatory active ingredient is in both the polymer phase and the complexing agent phase.

19. A method of hair removal comprising the steps of:
a) contacting an area of skin from which to remove hair with a substantially liquid depilatory composition comprising: (i) a polymer phase comprising a polymer capable of creating a hydrosol and complexing to form a hydrogel; (ii) a complexing agent phase comprising a complexing agent that complexes said polymer and creates a hydrogel from the hydrosol of said polymer; (iii) a depilatory active ingredient; and iv) a polyol;
b) complexing the polymer phase to form a pliable film hydrogel; and
c) removing said pliable film from contact with skin.

20. The method of claim 19 wherein the polymer is polyvinyl alcohol.

21. The method of claim 19 wherein the complexing agent is borax.
